(19) United States
(12) Patent Application Publication
(10) Pub. No.: US 2024/0142421 A1
(43) Pub. Date: May 2, 2024

(54) TEST METHOD FOR GAS AND TEMPERATURE DISTRIBUTION IN A GOAF

(71) Applicant: CCTEG Shenyang Research Institute, Fushun (CN)

(72) Inventors: Depeng Zhang, Fushun (CN); Yuntao Liang, Fushun (CN); Yong Sun, Fushun (CN); Junjie Zhang, Fushun (CN); Ruide Wang, Fushun (CN); Xinfeng Cui, Fushun (CN); Baolong Guo, Fushun (CN)

(21) Appl. No.: 17/974,425

(22) Filed: Oct. 26, 2022

(30) Foreign Application Priority Data
May 19, 2022 (CN) 202210540017.0

(51) Int. Cl. G01N 33/00; B65H 75/44; G01K 11/32

(52) U.S. Cl. CPC G01N 33/0027; B65H 75/4484; G01K 11/32; E21F 17/18

(57) ABSTRACT

The present disclosure includes a method of testing for gas and temperature distribution in a goaf, wherein the test device comprises a cable coupled to a cable dragging machine, the cable including a test end configured to be buried in the goaf; a gas temperature tester configured to test a gas for composition and temperature; and a gas pump configured to continuously pump gas from the test end and transmit it to the gas temperature tester. In some examples, the method comprises placing the test end of the cable in the goaf. According to some examples, the method comprises starting the cable dragging machine and starting the gas pump simultaneously with starting the cable dragging machine. The method may comprise withdrawing the cable, via the cable dragging machine, at a constant speed and analyzing, via the gas temperature tester, the composition and temperature of the gas.

### BACKGROUND

### Field

The present disclosure relates to the field of underground gas detection and analysis. Specifically, the present disclosure relates to gas detection and analysis in a coal mine or a goaf.

### Description of Related Art

Current methods of detecting gas and temperature distributions in a coal mine or goaf include embedding a beam pipe and a temperature sensor in advance. The devices for gas detection and temperature detection are independent, and installed in the inlet and return airways of the working face. As the working face advances, the devices for gas detection and temperature detection gradually enter the goaf, collecting gas in increments of time, such as every day or every working shift, in order to analyze the gas in a laboratory above ground while also testing the temperature.

Because the collected gas needs to be transported to the surface, the cycle time for the gas detection becomes longer as work is advanced further into the goaf. This longer cycle time means that the gas needs to be cleaned repeatedly prior to loading it into an air bag into order to minimize error. Additionally, to prevent the beam pipes and temperature conductors from being damaged by falling debris, iron or steel pipes are needed for protection. Because of these issues, these methods necessitate considerable manpower, material resources, and time. Thus, there is a need for systems and methods to remedy these deficiencies found in the prior art.

### SUMMARY

The present disclosure includes a method of testing for gas and temperature distribution in a goaf, wherein a test device comprises a cable coupled to a cable dragging machine, the cable including a test end configured to be buried in the goaf; a gas temperature tester configured to test a gas for composition and temperature; and a gas pump configured to continuously pump gas from the test end and transmit it to the gas temperature tester. In some examples, the method comprises placing the test end of the cable in the goaf. According to some examples, the method comprises starting the cable dragging machine. The method may comprise starting the gas pump simultaneously with starting the cable dragging machine. In some examples, the method comprises withdrawing the cable, via the cable dragging machine, at a constant speed. According to some examples, the method comprises analyzing, via the gas temperature tester, the composition and temperature of the gas.

The method may further comprise stopping, automatically, the cable dragging machine when the test end of the cable has been removed from the goaf. In some examples, the method further comprises stopping the gas pump simultaneously with stopping the cable dragging machine.

According to some examples, the method further comprises comparing a time when the gas enters the test end of the cable with the time when the gas enters the gas temperature tester. The method may further comprise determining a gas distribution in the goaf based on the composition of the gas and the time at which that composition of gas entered the test end of the cable. In some examples, the method further comprises determining a temperature distribution in the goaf based on the temperature of the gas and the time at which that temperature of gas entered the test end of the cable.

According to some examples, the test device further comprises a gas pipe coupled within the cable. The method of testing for gas and temperature distribution in a goaf may further comprises calculating a total gas transmission volume of the gas pipe based on an inner diameter of the gas pipe and a length of the gas pipe. In some examples, the method further comprises calculating a flow rate of the gas based on the inner diameter of the gas pipe, the length of the gas pipe, and a gas pump flow. According to some examples, the method further comprises calculating a gas venting time based on the total gas transmission volume and the flow rate of the gas.

The method may further comprise determining, via the flow rate of the gas, a withdrawal speed of the cable. In some examples, the method further comprises determining, via the withdrawal speed of the cable, a relationship between a withdrawal distance of the gas pipe and a withdrawal time of the gas pipe. According to some examples, the method further comprises establishing a correspondence between the time when the gas enters the test end of the cable and the time when the gas enters the gas temperature tester based on the relationship between the withdrawal distance of the gas pipe and the withdrawal time of the gas pipe.

Calculating the total gas transmission volume of the gas pipe may be calculated as:

$$\\ V = π {L ( \frac{d}{2} * {10}^{- 3} )}^{2}$$

where V is the total gas transmission volume of the gas pipe, L is the length of the gas pipe and d is the inner diameter of the gas pipe. In some examples, calculating the flow rate of the gas is calculated as:

$$\\ V = \frac{240 q}{π {d}^{2}}$$

where v is the flow rate of the gas, and q is the gas pump flow. According to some examples, calculating the gas venting time is calculated as:

$$\\ {t}_{0} = \frac{V}{q} = \frac{L}{v} = \frac{L π {d}^{2}}{240 q}$$

where t₀ is the gas venting time. Determining the withdrawal speed of the cable may be calculated as:

v₁=πnD*10⁻³

where v₁ is the withdrawal speed of the cable, n is a rotational speed of a motor of the cable dragging machine, and D is an outer diameter of a motor shaft of the cable dragging machine. In some examples, determining the relationship between the withdrawal distance of the gas pipe and the withdrawal time of the gas pipe is calculated as:

$${s}_{0} = {v}_{1} t = π nDt * {10}^{- 3} = \frac{240 qt}{π {d}^{2}}$$

where s₀ is the withdrawal distance of the cable and t is the withdrawal time. According to some examples, the length of the cable in the goaf at a certain time point is calculated as:

$$L - {s}_{0} = L - 0.001 π nDt = L - \frac{240 qt}{π {d}^{2}}$$

and, establishing a correspondence between the time when the gas enters the test end of the cable and the time when the gas enters the gas temperature tester is calculated as:

$${t}_{c} = t - {t}_{0} = t - \frac{L π {d}^{2}}{240 q}$$

where t_{c} is the time when the gas enters the gas temperature tester, t is the time when the gas enters the test end of the cable, and t₀ is the time when the cable dragging machine begins to drag.

The method of testing for gas and temperature distribution in a goaf may further comprise transmitting information on the distribution of composition and temperature of the gas in the goaf to an external computer for data aggregation and analysis. In some examples, the method further comprises clarifying the coal spontaneous combustion (CSC) “three zones” and gas distribution areas in the goaf.

According to some examples, the method further comprises transmitting information on the distribution of composition and temperature of the gas in the goaf to an external computer for data aggregation and analysis. The method may further comprise clarifying the coal spontaneous combustion (CSC) “three zones” and gas distribution areas in the goaf.

In some examples, the cable further comprises a temperature measuring optical fiber, wherein analyzing the temperature of the gas further comprises converting a temperature detection signal from an optical fiber signal to an electrical signal.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIG. 1 illustrates a schematic diagram showing the connection of gas and temperature distribution test devices, according to some examples.

FIG. 2 illustrates a schematic circuit diagram of a gas temperature tester, according to some examples.

FIG. 3A illustrates a side view of a wire spool, according to some examples.

FIG. 3B illustrates a side view of an additional exemplary wire spool from FIG. 3A.

FIG. 4 illustrates a cross-sectional front view of the wire spool of either FIG. 3A or FIG. 3B, according to some examples.

FIG. 5A illustrates a cross-sectional front view of a cable as might be included in FIG. 3A, according to some examples.

FIG. 5B illustrates a cross-sectional front view of a cable as might be included in FIG. 3B, according to some examples.

FIG. 6 illustrates a flow chart depicting a method of using a cable to test for gas and temperature distribution in a goaf, according to some examples.

FIG. 7 illustrates a flow chart depicting a method of calculating various facets of a gas in a goaf, according to some examples.

FIG. 8 illustrates a flow chart depicting a method of determining a speed of a gas in a goaf, according to some examples.

FIG. 9 illustrates a flow chart depicting a method of aggregating data about gas in a goaf, according to some examples.

### DETAILED DESCRIPTION

Although specific embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order-dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### COMPONENT INDEX

10—Test device
20—Gas temperature tester
30—Gas pump
40—Connector
50—Wire spool
60—Cable
70—Computer
80—Goaf
202—Single-chip microcomputer
204—Audible and visual alarm module
206—Display
208—Storage module
210—Optical fiber conversion module
212—Power supply module
214—Gas sensor
302—Length counter
304—Support
306—Roll
308—Baffle
310—Handle
312—Explosion-proof motor
314—Universal wheel
402—Test end
404—Output end
502—Gas pipe
504—Water insulation layer
506—Flame-retardant plastic
508—Cable paste
510—Stranded steel wire
512—Temperature measuring optical fiber

At different stages of oxidation during spontaneous combustion of coal, there are corresponding spontaneous combustion temperature ranges, types of gaseous products, and concentrations of gaseous products. Among these gaseous products some are used to predict the process of coal spontaneous combustion (CSC), including O₂, CO, CO₂, CH₄, C₂H₆, C₃H₈, C₂H₄, C₃H₆, C₂H₂, etc. In light of the composition, concentration, and distribution characteristics of oxidized gaseous products, the CSC “three zones” in the goaf can be accurately divided and predicted, which serves to guide safe production, fire control, and management in the coal mine. The indexes used to divide the CSC “three zones” primarily include temperature, O₂, CO, CO₂, C₂H₄, and C₂H₂. Thus, in the field of coal safety technology, dividing the CSC “three zones” in a reasonable manner is of great significance and practicality for safe production in the coal mine, prevention of CSC, and secondary accidents. Consequently, research on rapid testing devices and methods of dividing the CSC “three zones” in the goaf is one of the most important means to ensure safe production within the coal mine and prevent CSC.

Due to low airflow velocity in the goaf, a large amount of CH₄ cannot be diluted in a quick enough manner, which leads to accumulation of this gas. Once the CH₄ makes contact with fire, likely from an external source, gas explosions, gas combustion, or other accidents become more likely to occur. Furthermore, gas drainage distance in goaf is generally determined based on experience of an operator—commonly, this distance is between 20 and 40 meters. Thus, measurement of the distribution of CH₄ is also significant to determine the range of gas drainage in the goaf in order to remove reliance on an operator, which can take human error out of the equation.

Current methods of detecting gas and temperature distributions in a coal mine or goaf include embedding a beam pipe and a temperature sensor in the inlet and return airways of the working face. As progress is made into the coal mine, the devices for gas detection and temperature detection also move further into the coal mine, collecting information at set intervals of time. After the gas is collected, it is brought to the surface to be tested in a laboratory. This collection of gas necessitates movement of the gas between the goaf and the above-ground laboratory, which becomes a longer and longer trip as progress is made into the coal mine. This longer trip means that the gas needs to be cleaned repeatedly prior to loading it into an air bag into order to minimize error. Additionally, to prevent the beam pipes and temperature conductors from being damaged by falling debris, protection, such as iron or steel pipe, is needed. Because of these issues, these methods necessitate considerable manpower, material resources and time.

Existing gas monitoring in a goaf is mainly applied in the drainage pipeline, and this monitoring experiences hysteresis. Additionally, the types of monitoring gasses are limited to Methylene (CH), Oxygen (O₂), Carbon Dioxide (CO₂), and Carbon Monoxide (CO), and, therefore, other gases produced during the oxidation of residual coal in the goaf cannot be fully detected. Finally, because the temperature sensor in the drainage pipeline is usually installed near the pipeline valve, comprehensive temperature detection cannot be realized further into the goaf.

To rectify the issues in the prior art, the present disclosure provides a test method for gas and temperature distribution in a coal mine or goaf. The present disclosure includes advantages such as a short detection cycle, direct and quick detection of gas and temperature underground, and removes the need to protect any element of the design from falling debris, such as through the use of iron or steel pipes.

The disclosure presented herein includes a test device for gas and temperature distribution in a goaf, which comprises a gas temperature tester, a gas pump, a connector, a wire spool, and a cable. The cable, which may be wound about the wire spool, includes a test end located on one side of the wire spool, which is configured to be buried within the goaf. Opposite the test end of the cable may be an output end. The output end of the cable may be coupled with the gas temperature tester through the connector and the gas pump, and it may be used to obtain gas and temperature detection signals at various test positions that are passed by the test end during withdrawal from the goaf. The output end then outputs these gas and temperature detection signals to the gas temperature tester once the test end has been withdrawn from the goaf.

This disclosure realizes the rapid testing of gas and temperature distribution in a goaf through cable withdrawal and analysis via a gas temperature tester. The disclosure overcomes the shortcomings of existing solutions (i.e., long analysis cycles, complex technology, the susceptible nature of analysis results due to the level of skill of the operators, etc.). Additionally, the disclosure facilitates the reduction of labor and time cost and achieves multi-point monitoring within a goaf. Through this multi-point monitoring, the disclosure achieves accurate analysis of the temperature, composition of gas, and concentration of gas within a goaf. This permits quick, accurate judgment about the state of development of potential spontaneous combustion within the goaf, which provides the basis for the control and management of this CSC. Furthermore, this disclosure achieves monitoring of the gas concentration in a goaf and provides a basis for the layout of a gas drainage pipeline, which is of great significance for facilitating safe production within the coal mine, and the safety of the miners.

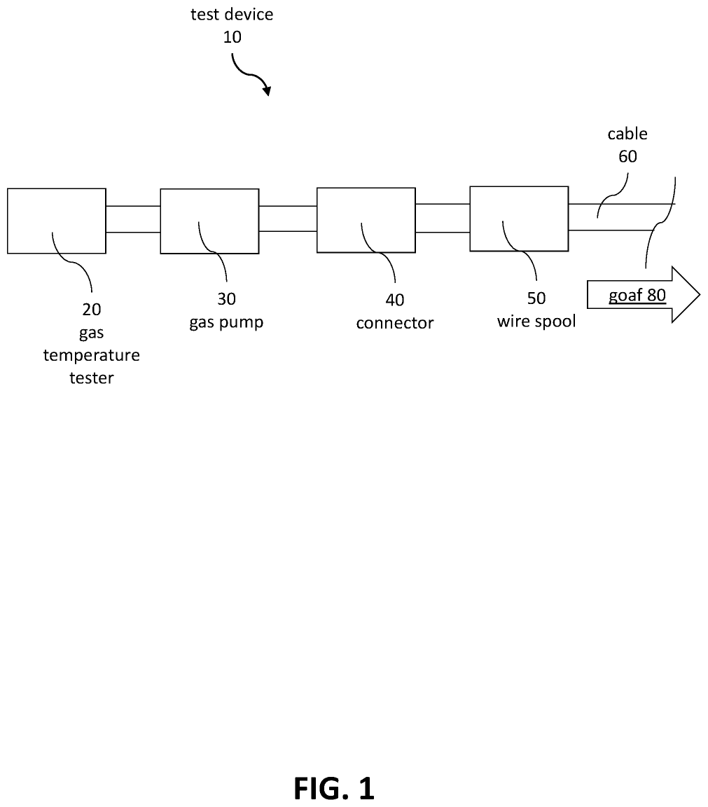

FIG. 1 illustrates a schematic diagram showing the connection of gas and temperature distribution test devices, As shown in FIG. 1, the test device for gas and temperature distribution may comprise, at least, a gas temperature tester, a gas pump, a connector, a wire spool, and a cable. The cable may be configured to wind about the wire spool. In some examples, the cable includes a test end located on one side of the wire spool and an output end located on the side of the wire spool opposite the test end. The test end may be configured to be buried in a goaf, and the output end may be configured to couple to the gas temperature tester through the connector and the gas pump. According to some examples, as the wire spool rotates, the test end is withdrawn from the goaf and through the coal mine. During this withdrawal, the output end of the cable may be used to obtain gas and temperature detection signals at various testing positions that are passed by the test end. The output end of the cable may then communicate this information, or output it, to the gas temperature tester.

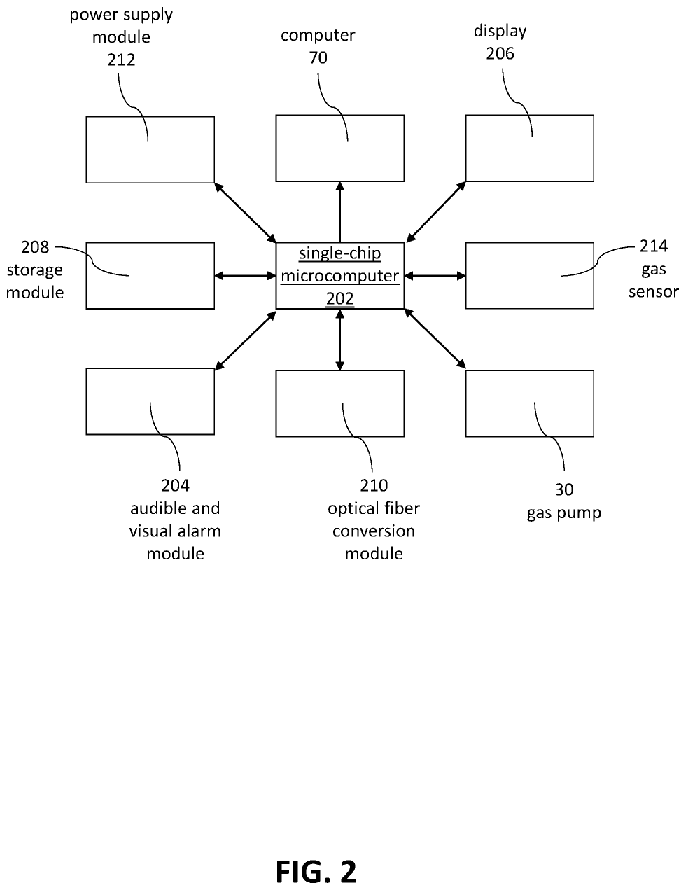

The gas temperature tester may also be used to analyze the gas composition and temperature corresponding with each testing position. As illustrated in FIG. 2, the gas temperature tester may include a single-chip microcomputer. According to some examples, the gas temperature tester comprises a gas sensor electrically coupled to the single-chip microcomputer and configured to detect the composition of supplied gaseous information. In some examples, the gas temperature tester includes an optical fiber conversion module electrically coupled to the single-chip microcomputer and configured to convert a temperature detection signal from an optical fiber signal to an electrical signal. The single-chip microcomputer may also be electrically coupled to the gas pump and configured to control the start and stop of the gas pump. The gas temperature tester may also include a gas path coupled to the output end of the cable and configured to transmit the gas output by the cable to the gas sensor.

FIG. 2 illustrates a schematic circuit diagram of a gas temperature tester. As seen in FIG. 2, the single-chip microcomputer may also be communicatively coupled to a storage module for storing information on the gas composition and temperature. In some examples, the single-chip microcomputer is electrically coupled to an audible and visual alarm module for alarming a user as to when the gas temperature tester breaks down and/or the gas concentration exceeds an upper threshold limit. According to some examples, the single-chip microcomputer electrically couples to a display that is configured to display information pertaining to the composition and temperature of the gas. The single-chip microcomputer may be further electrically coupled to a power supply configured to supply power to the gas temperature tester as a whole.

In some examples, the gas enters the gas path through the cable as a consequence of the performance of the gas pump. The gas then may enter the gas sensor, which is in communication with the single-chip microcomputer, for analysis of the composition and temperature of the gas. According to some examples, this analysis data is then displayed on the display before being stored in the storage module.

The temperature detection signal may enter the optical fiber conversion module from the temperature measuring optical fiber of the cable. Here, an optical fiber signal indicative of the temperature detection signal may be converted into an electrical signal. Through instruction from the single-chip microcomputer, the temperature detection signal may help to perform the temperature test and display the resulting test data through the display before storing the data in the storage module. The single-chip microcomputer is not proprietary and may be a commercially available product that is preprogrammed for use with the gas temperature tester.

In some examples, the power supply module comprises a transformer and a rectifier module. The primary side of the transformer may be coupled to the electric supply through a switch, and the secondary side of the transformer may be coupled with the input end of the rectifier module. According to some examples, the output end of the rectifier module is coupled with each electrical component of the gas temperature tester to enable power from the power supply to reach each of these components.

Similarly, the gas sensor may be non-proprietary and commercially available. Depending on the needs of the user, many different commercial products for detecting gas composition, such as a CO sensor, a Methane (CH₄) Sensor, an O₂ sensor, a CO₂ sensor, an Ethylene (C₂H₄) sensor, or an Acetylene (C₂H₂) sensor may be used in conjunction with the gas temperature tester.

The single-chip microcomputer may also be coupled to an external computer. In some examples, the external computer is configured to determine the distribution law of gas and temperature according to the gas composition and gas temperature at each test position that is uploaded by the single-chip microcomputer.

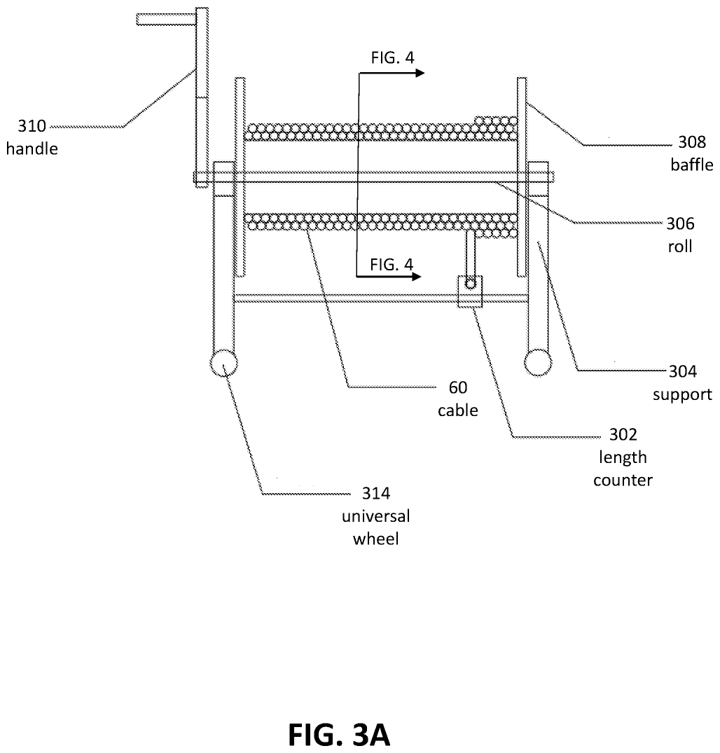

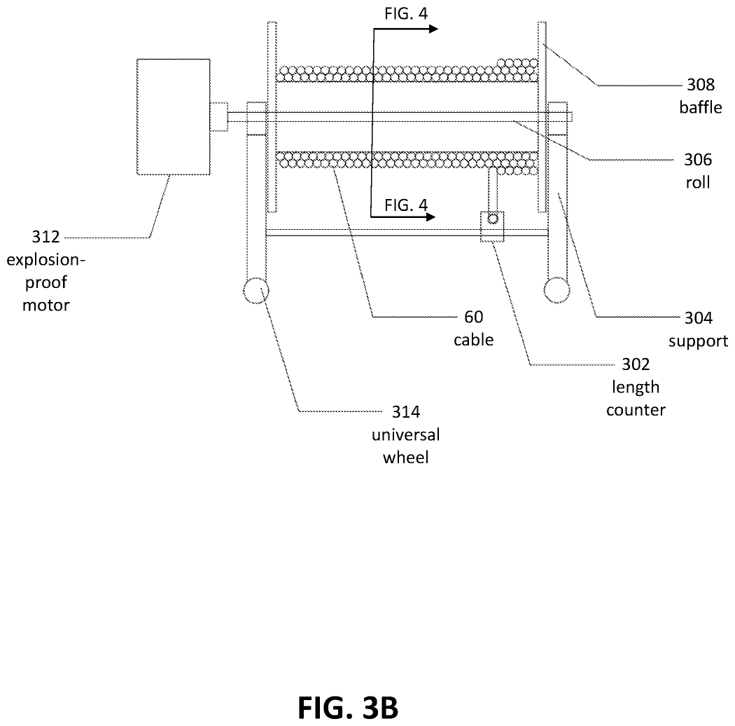

FIGS. 3A and 3B illustrate side views of a wire spool according to two examples. As can be seen in FIGS. 3A and 3B, the wire spool may include a support with a rotatable roll that includes two baffles, one on either side of the roll. The cable may be configured to wind on the roll between the two baffles, which allows for the picking up and laying down of the cable through rotation of the roll. In some examples, in order to facilitate transport of the gas temperature tester as a whole, at least one universal wheel is coupled to the bottom of the support to allow lateral movement of the system.

As can be seen in FIG. 3A, one of the rolls may be coupled to a handle. An operator may turn the handle, which will actuate the rotation of the roll. In this manner, the operator may manually recover the cable. The baffle is used to fix the cable in place on the roll, so that the cable does not slip off either end during the winding process. The universal wheel installed at the bottom of the support may facilitate movement of the system as a whole, and permit dragging of the wire spool in any direction.

Alternatively, as shown in FIG. 3B, the end of the roll that is coupled to a handle may instead be coupled to an explosion-proof motor. This explosion-proof motor may facilitate and control the rotation of the roll, which can help to save manpower as no operator need be present for manual rotation of the roll. The explosion-proof motor may also be electrically coupled to the single-chip microcomputer of the gas temperature tester, allowing the single-chip microcomputer to automatically control the start and stop of the explosion-proof motor, which in turn either starts or stops the rotation of the wire spool.

According to some examples, when the gas temperature tester receives the gas detection signals and optical fiber signals and obtains data from the test positions after the analysis is completed, the explosion-proof motor will automatically restart until the test end is withdrawn completely from the goaf. This allows for a periodic control cycle, which can further save on necessary manpower.

As shown in both FIGS. 3A and 3B, the device may comprise a length counter coupled to the cable. In some examples, the length counter is installed on the support in the wire spool and is configured to detect the length of withdrawal of the test end of the cable so that a positional relationship between the test end and each testing position can be determined. This length counter may be usable with other inventions within this field of technology to facilitate the detection of withdrawal lengths of cables in a coal mine.

According to some examples of this disclosure, the cable includes the temperature measuring optical fiber and the gas pipe. The temperature measuring optical fiber may be used to output the temperature detection signal to the gas temperature tester. In some examples, the gas pipe is used to output the gas from the position of the test end of the cable to the gas temperature tester.

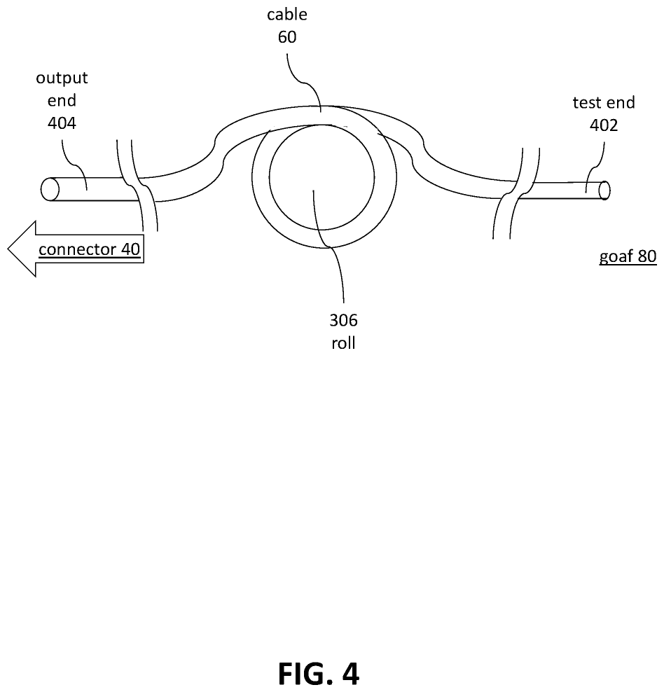

FIG. 4 illustrates a cross-sectional front view of the wise spool of either FIG. 3A or FIG. 3B, according to some examples. FIG. 4 illustrates the test end 402 being located within the goaf 80 (at the beginning of testing). The cable 60 leads from the test end 402 to a roll 306, where the cable 60 is wound as previously discussed. The cable 60 then continues to the output end 404, which may be coupled to a connector 40.

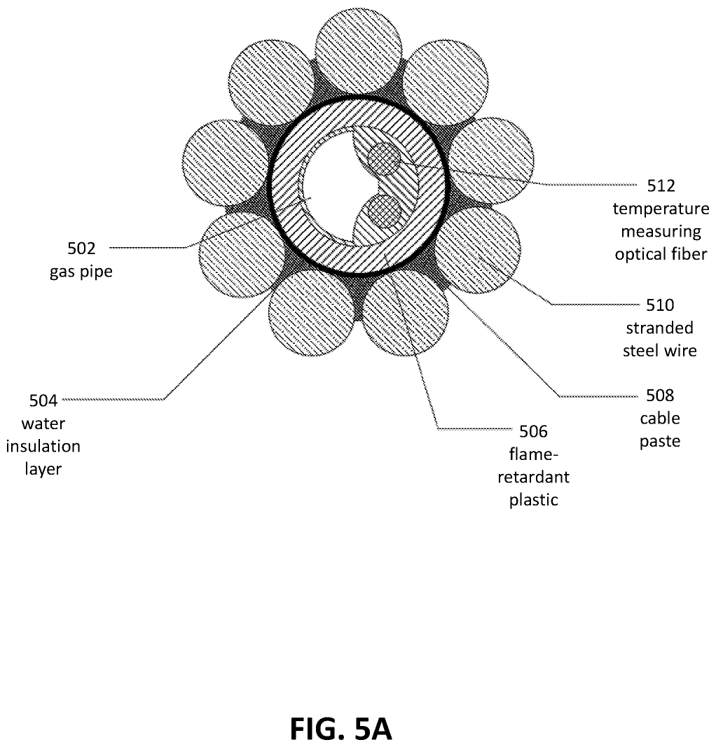

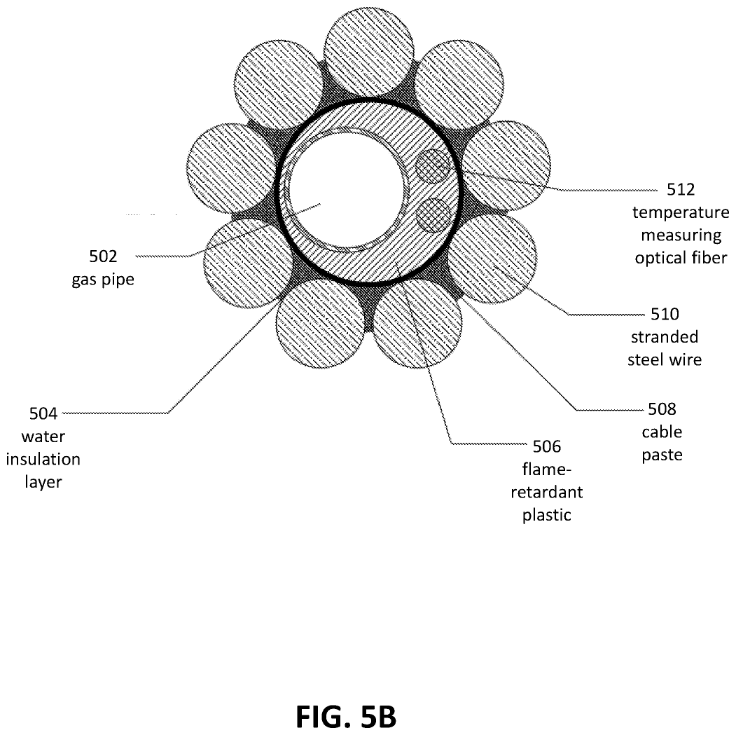

FIG. 5A illustrates a cross-sectional front view of a cable as might be included in FIG. 3A, and FIG. 5B illustrates a cross-sectional front view of a cable as might be included in FIG. 3B. As can be seen in both FIGS. 5A and 5B, the cable may comprise a water insulation layer, cable paste, and stranded steel write. In some examples, the gas pipe is wrapped with flame retardant plastic. According to some examples, the temperature-measuring optical fiber is arranged in the gas pipe and/or the flame-retardant plastic surrounding the gas pipe.

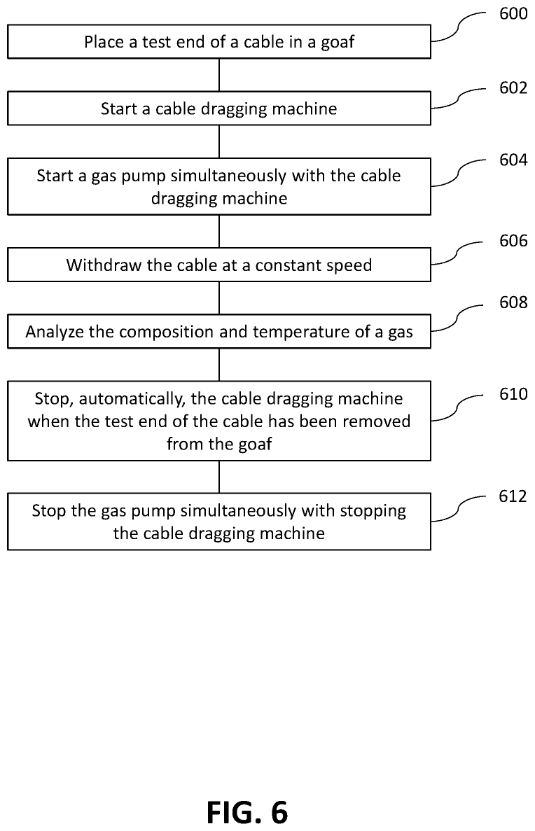

FIG. 6 illustrates a flow chart depicting a method of using a cable to test for gas and temperature distribution in a goaf, according to some examples. In some examples, the method includes placing a test end of a cable in a goaf (at step 600). According to some examples, the method includes starting a cable dragging machine (at step 602). The method may include starting a gas pump simultaneously with the cable dragging machine (at step 604). In some examples, the method includes withdrawing the cable at a constant speed (at step 606). According to some examples, the method includes analyzing the composition and temperature of a gas (at step 608). The method may include stopping, automatically, the cable dragging machine when the test end of the cable has been removed from the goaf (at step 610). In some examples, the method includes stopping the gas pump simultaneously with stopping the cable dragging machine (at step 612).

While not depicted in the flow chart of FIG. 6, analyzing the temperature of the gas may comprise converting a temperature detection signal from an optical fiber signal to an electrical signal, in exemplary systems where the cable comprises a temperature measuring optical fiber

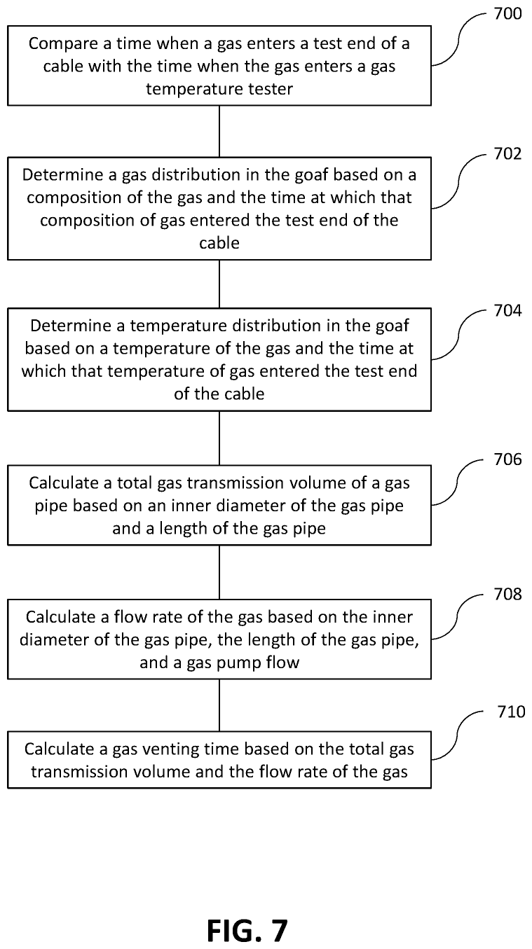

FIG. 7 illustrates a flow chart depicting a method of calculating various facets of a gas in a goaf, according to some examples. In some examples, the method includes comparing a time when a gas enters a test end of a cable with the time when the gas enters a gas temperature tester (at step 700). According to some examples, the method includes determining a gas distribution in the goaf based on a composition of the gas and the time at which that composition of gas entered the test end of the cable (at step 702). The method may include determining a temperature distribution in the goaf based on a temperature of the gas and the time at which that temperature of gas entered the test end of the cable (at step 704). In some examples, the method includes calculating a total gas transmission volume of a gas pipe based on an inner diameter of the gas pipe and a length of the gas pipe (at step 706). According to some examples, the method includes calculating a flow rate of the gas based on the inner diameter of the gas pipe, the length of the gas pipe, and a gas pump flow (at step 708). The method may include calculating a gas venting time based on the total gas transmission volume and the flow rate of the gas (at step 710).

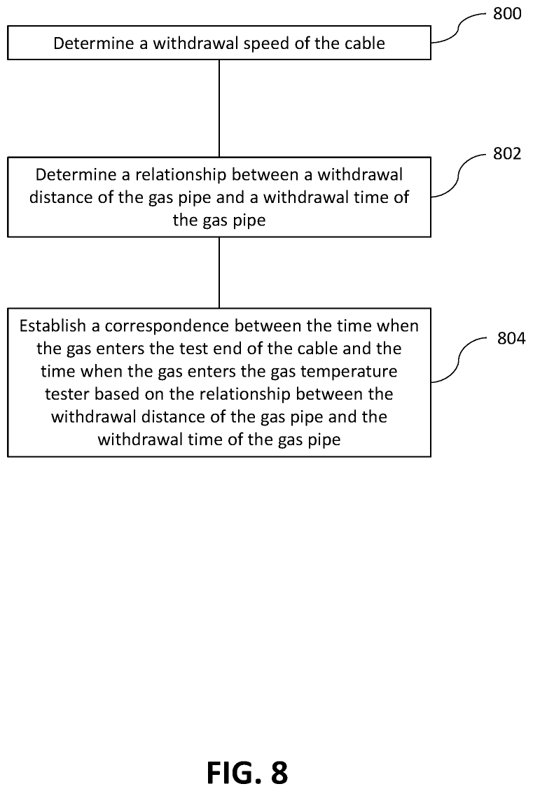

FIG. 8 illustrates a flow chart depicting a method of determining a speed of a gas in a goaf, according to some examples. In some examples, the method includes determining a withdrawal speed of the cable (at step 800). According to some examples, the method includes determining a relationship between a withdrawal distance of the gas pipe and a withdrawal time of the gas pipe (at step 802). The method may include establishing a correspondence between the time when the gas enters the test end of the cable and the time when the gas enters the gas temperature tester based on the relationship between the withdrawal distance of the gas pipe and the withdrawal time of the gas pipe (at step 804).

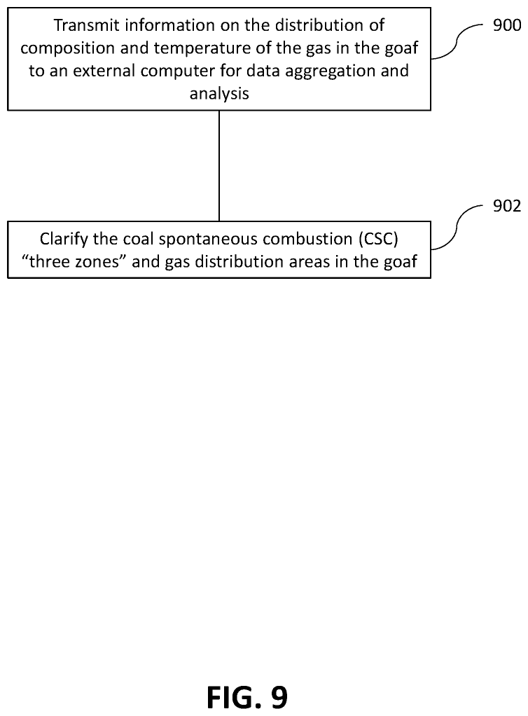

FIG. 9 illustrates a flow chart depicting a method of aggregating data about gas in a goaf, according to some examples. In some examples, the method includes transmitting information on the distribution of composition and temperature of the gas in the goaf to an external computer for data aggregation and analysis (at step 900). According to some examples, the method includes clarifying the coal spontaneous combustion (CSC) “three zones” and gas distribution areas in the goaf (at step 902).

The above systems and methods are guided by a series of equations detailed herein. Calculating the total gas transmission volume of the gas pipe is calculated as:

$$V = π {L ( \frac{d}{2} * {10}^{- 3} )}^{2}$$

where V is the total gas transmission volume of the gas pipe, L is the length of the gas pipe and d is the inner diameter of the gas pipe.

Calculating the flow rate of the gas is calculated as:

$$v = \frac{q}{{π ( \frac{d}{2} )}^{2}}$$

where v is the flow rate of the gas, and q is the gas pump flow.

Calculating the gas venting time is calculated as:

$${t}_{0} = \frac{V}{q} = \frac{L}{v} = \frac{15 L π {d}^{2}}{q}$$

where t₀ is the gas venting time.

Determining the withdrawal speed of the cable is calculated as:

v₁=πnD*10⁻³

where v₁ is the withdrawal speed of the cable, n is a rotational speed of the motor of the cable dragging machine, and D is an outer diameter of a motor shaft of the cable dragging machine.

Determining the relationship between the withdrawal distance of the gas pipe and the withdrawal time of the gas pipe is calculated as:

$${s}_{0} = {v}_{1} t = π nDt * {10}^{- 3} = \frac{240 qt}{π {d}^{2}}$$

where s₀ is the withdrawal distance of the cable and t is the withdrawal time.

The length of the cable in the goaf at a certain time point is calculated as:

$$L - {s}_{0} = L - 0.001 π nDt = L - \frac{240 qt}{π {d}^{2}}$$

and establishing a correspondence between the time when the gas enters the test end of the cable and the time when the as enters the gas temperature tester is calculated as:

$${t}_{c} = t - {t}_{0} = t - \frac{15 L π {d}^{2}}{q}$$

where t_{c} is the time when the gas enters the gas temperature tester, t is the time when the gas enters the test end of the cable, and t₀ is the time when the cable dragging machine begins to drag.

### INTERPRETATION

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1, and embodiments described in other sections may apply to and be combined with embodiments described within the “Topic 1” section.

To increase the clarity of various features, other features are not labeled in each figure.

The various features and processes described above may be used independently of one another or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, parallel, or some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless expressly stated otherwise, or otherwise understood within the context as used, is generally intended to convey that certain embodiments include, while other embodiments do not include, certain features, elements and/or steps. Thus, such conditional language is not generally intended to imply that features, elements, and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless expressly stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

While certain example embodiments have been described, these embodiments have been presented by way of example only and are not intended to limit the scope of the inventions disclosed herein. Thus, nothing in the foregoing description implies that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

What is claimed is:

1. A method of testing for gas and temperature distribution in a goaf, wherein a test device comprises a cable coupled to a cable dragging machine, the cable including a test end configured to be buried in the goaf; a gas temperature tester configured to test a gas for composition and temperature; and a gas pump configured to continuously pump gas from the test end and transmit it to the gas temperature tester; the method comprising:
placing the test end of the cable in the goaf;
starting the cable dragging machine;
starting the gas pump simultaneously with starting the cable dragging machine;
withdrawing the cable, via the cable dragging machine, at a constant speed; and
analyzing, via the gas temperature tester, the composition and temperature of the gas.

2. The method of claim 1, further comprising:
stopping, automatically, the cable dragging machine when the test end of the cable has been removed from the goaf; and
stopping the gas pump simultaneously with stopping the cable dragging machine.

3. The method of claim 1, further comprising comparing a time when the gas enters the test end of the cable with the time when the gas enters the gas temperature tester.

4. The method of claim 3, further comprising:
determining a gas distribution in the goaf based on the composition of the gas and the time at which that composition of gas entered the test end of the cable; and
determining a temperature distribution in the goaf based on the temperature of the gas and the time at which that temperature of gas entered the test end of the cable.

5. The method of claim 3, the test device further comprising a gas pipe coupled within the cable, the method further comprising:
calculating a total gas transmission volume of the gas pipe based on an inner diameter of the gas pipe and a length of the gas pipe;
calculating a flow rate of the gas based on the inner diameter of the gas pipe, the length of the gas pipe, and a gas pump flow; and
calculating a gas venting time based on the total gas transmission volume and the flow rate of the gas.

6. The method of claim 5, further comprising:
determining, via the flow rate of the gas, a withdrawal speed of the cable; and
determining, via the withdrawal speed of the cable, a relationship between a withdrawal distance of the gas pipe and a withdrawal time of the gas pipe.

7. The method of claim 6, further comprising establishing a correspondence between the time when the gas enters the test end of the cable and the time when the gas enters the gas temperature tester based on the relationship between the withdrawal distance of the gas pipe and the withdrawal time of the gas pipe.

8. The method of claim 7, wherein calculating the total gas transmission volume of the gas pipe is calculated as:
$$V = π {L ( \frac{d}{2} * {10}^{- 3} )}^{2}$$
where V is the total gas transmission volume of the gas pipe, L is the length of the gas pipe and d is the inner diameter of the gas pipe.

9. The method of claim 8, wherein calculating the flow rate of the gas is calculated as:
$$v = \frac{q}{{π ( \frac{d}{2} )}^{2}}$$
where v is the flow rate of the gas, and q is the gas pump flow.

9. The method of claim 9, wherein calculating the gas venting time is calculated as:
$${t}_{0} = \frac{V}{q} = \frac{L}{v} = \frac{15 L π {d}^{2}}{q}$$
where t₀ is the gas venting time.

11. The method of claim 10, wherein determining the withdrawal speed of the cable is calculated as:
v₁=πnD*10⁻³
where v₁ is the withdrawal speed of the cable, n is a rotational speed of a motor of the cable dragging machine, and D is an outer diameter of a motor shaft of the cable dragging machine.

12. The method of claim 11, wherein determining the relationship between the withdrawal distance of the gas pipe and the withdrawal time of the gas pipe is calculated as:
$${s}_{0} = {v}_{1} t = π nDt * {10}^{- 3} = \frac{240 qt}{π {d}^{2}}$$
where s₀ is the withdrawal distance of the cable and t is the withdrawal time.

13. The method of claim 12, wherein the length of the cable in the goaf at a certain time point is calculated as:
$$L - {s}_{0} = L - 0.001 π nDt = L - \frac{240 qt}{π {d}^{2}}$$
and,
wherein establishing a correspondence between the time when the gas enters the test end of the cable and the time when the gas enters the gas temperature tester is calculated as:
$${t}_{c} = t - {t}_{0} = t - \frac{15 L π {d}^{2}}{q}$$
where t_{c} is the time when the gas enters the gas temperature tester, t is the time when the gas enters the test end of the cable, and t₀ is the time when the cable dragging machine begins to drag.

14. The method of claim 2, further comprising transmitting information on the distribution of composition and temperature of the gas in the goaf to an external computer for data aggregation and analysis.

15. The method of claim 14, further comprising clarifying the coal spontaneous combustion (CSC) “three zones” and gas distribution areas in the goaf.

16. The method of claim 3, further comprising transmitting information on the distribution of composition and temperature of the gas in the goaf to an external computer for data aggregation and analysis.

17. The method of claim 16, further comprising clarifying the coal spontaneous combustion (CSC) “three zones” and gas distribution areas in the goaf.

18. The method of claim 1, wherein the cable further comprises a temperature measuring optical fiber, wherein analyzing the temperature of the gas further comprises converting a temperature detection signal from an optical fiber signal to an electrical signal.
